(19) United States
(12) Patent Application Publication
(10) Pub. No.: US 2009/0203991 A1
(43) Pub. Date: Aug. 13, 2009

(54) MULTIPLE IMAGING AND/OR SPECTROSCOPIC MODALITY PROBE

(75) Inventors: Thanassis Papaioannou, Los Angeles, CA (US); Qiyin Fang, Los Angeles, CA (US); Javier A. Jo, Los Angeles, CA (US); Laura Marcu, Sierra Madre, CA (US); K. Kirk Shung, Monterey Park, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignees: CEDARS-SINAI MEDICAL CENTER, Los Angeles, CA (US); UNIVERSITY OF SOUTHERN CALIFORNIA, Los Angeles, CA (US)

(21) Appl. No.: 12/297,080

(22) PCT Filed: Apr. 21, 2006
(86) PCT No.: PCT/US06/14988

§ 371 Date: Oct. 14, 2008

(51) Int. Cl. A61B 5/055; A61B 1/00; A61B 8/14; A61B 6/00

(52) U.S. Cl. 600/421; 600/104; 600/462; 600/478

(57) ABSTRACT

The apparatus and methods described herein enable an operator to simultaneously collect images and spectroscopic information from a region(s) of interest using a multiple modality imaging and/or spectroscopic probe, configured as a catheter, endoscope, microscope, or hand held probe. The device may incorporate, for example, an ultrasonic transducer and a fiber optic probe to translate images and spectra. The apparatus and methods may be used in any suitable cavity, for example, the vascular system of a mammal.

### FIELD OF THE INVENTION

The present invention relates to an apparatus and method for the collection of images and/or spectroscopic information from a region(s) of interest. More specifically, the method and apparatus of the present invention allow for simultaneous low and high spatial resolution probing and for simultaneous imaging and spectroscopic analysis.

### BACKGROUND OF THE INVENTION

Despite significant progress in treatment of atherosclerotic cardiovascular disease, it results in more than 19 millions deaths annually. A large number of victims of the disease die suddenly—without prior symptoms—due to plaque rupture. This critical event, to a large extent, has been associated with “vulnerable” plaque. Existing screening and diagnostic methods are insufficient to identify the victims before the event occurs. (Libby, P. et al., “Stabilization of atherosclerotic plaques: new mechanisms and clinical targets,” Nat Med. 8:1257-1262 (2002)). Most techniques identify luminal diameter or stenosis, wall thickness, and plaque volume, but are inefficient in identifying the rupture-prone plaque. (Id.; Naghavi, M. et al., “From vulnerable plaque to vulnerable patient—A call for new definitions and risk assessment strategies: Part I,” Circulation 108:1664-1672 (2003); MacNeill, S D et al., “Intravascular modalities for detection of vulnerable plaque—Current status,” Arteriosclerosis Thrombosis and Vascular Biology 23:1333-1342 (2003)). New diagnostic techniques to localize and characterize vulnerable plaques are needed. Many intravascular diagnostic techniques have been proposed for assessment of plaque vulnerability including nuclear magnetic resonance (NMR) spectroscopy (Tearney, G. J. et al., “Quantification of macrophage content in atherosclerotic plaques by optical coherence tomography,” Circulation 107:113-119 (2003); Jang, I. K. et al., “Visualization of tissue prolapse between coronary stent struts by optical coherence tomography—Comparison with intravascular ultrasound,” Circulation 104:2754 (2001); Hatsukami, T. S. et al., “Visualization of fibrous cap thickness and rupture in human atherosclerotic carotid plaque in vivo with high-resolution magnetic resonance imaging,” Circulation 102:959-964 (2000); Zimmermann, G. G. et al., “Intravascular MR imaging of atherosclerotic plaque: Ex vivo analysis of human femoral arteries with histologic correlation,” Radiology 204:769-774 (1997)), intravascular ultrasound (IVUS—including high-frequency, elastography) (de Korte, C. L. et al., “Morphological and mechanical information of coronary arteries obtained with intravascular elastography—Feasibility study in vivo,” European Heart Journal 23:405-413 (2002); Jang I. K., et al., “Comparison of optical coherence tomography and intravascular ultrasound for detection of coronary plaques with large lipid-core in living patients,” Circulation 102:509 (2000); Maruvada, S. et al., “High-frequency backscatter and attenuation measurements of porcine erythrocyte suspensions between 30-90 MHz,” Ultrasound in Medicine and Biology 28:1081-1088 (2002); Yock, P. G. et al., “Intravascular ultrasound: State of the art and future directions,” American Journal of Cardiology 81:27 E-32E (1998)), optical coherence tomography (OCT) (Bouma, B. E. et al., “Evaluation of intracoronary sterling by intravascular optical coherence tomography,” Heart 89:317-320 (2003); Fujimoto, J. G. et al., “High resolution in vivo intra-arterial imaging with optical coherence tomography,” Heart 82:128-133 (1999)), thermography (Verheye, S. et al., “In vivo temperature heterogeneity of atherosclerotic plaques is determined by plaque composition,” Circulation 105:1596-1601 (2002); Casscells, W. et al., “Thermal detection of cellular infiltrates in living atherosclerotic plaques: possible implications for plaque rupture and thrombosis,” Lancet 347:1447-1451 (1996); Stefanadis, C. et al., “Statin treatment is associated with reduced thermal heterogeneity in human atherosclerotic plaques,” Eur Heart J. 23:1664-1669 (2002)), and spectroscopic methods (MacNeill, B. D. et al., “Intravascular modalities for detection of vulnerable plaque —Current Status,” Arteriosclerosis Thrombosis and Vascular Biology 23:1333-1342 (2003); Moreno, P. R. et al., “Identification of high-risk atherosclerotic plaques: a survey of spectroscopic methods,” Curr Opin Cardiol 17:638-647 (2002)). Several forms of optical spectroscopy have been used in atherosclerosis research including Raman, near-infrared (NIR), diffuse reflectance NIR, and fluorescence spectroscopy.

Diagnostic imaging and spectroscopic techniques are powerful medical, veterinary and industrial tools that allow physicians and other practitioners to explore bodily structures and functions and remote spaces with a minimum of invasion. Indeed, advances in diagnostic technology have allowed physicians, for example, to evaluate processes and events as they occur in vivo. Technological innovations have opened the door for the development and widespread use of sonic (ultrasound) and magnetic resonance imaging (MRI), X-ray imaging, fluorescent screens, nuclear magnetic resonance (NMR), computed tomography (CT), positive emission tomography (P.E.T.), and endoscopic techniques, which allow physicians, for example, to accurately and efficiently diagnose pathology. Diagnostic tools allow physicians, for example, to use image-guided surgical methods to more accurately determine the locations of tumors, lesions, and a host of vascular abnormalities. Moreover, innovations in computer technology and imaging when used in conjunction with optical, electromagnetic, or ultrasound sensors allow physicians to make real-time diagnosis a part of surgical procedures.

Ultrasonic imaging is a mature medical, veterinary and industrial technology that accounts for one in four imaging studies. Ultrasonic devices produce high frequency sound waves that are able to penetrate the surface of a target and reflect off internal target structures and these techniques are used to identify, for example, pathology related to blood flow (arteriosclerosis). In various applications, microscopes can be configured to use ultrasound to study cell structures without subjecting them to lethal staining procedures that can also impede diagnosis through the production of artifacts. One ultrasonic application, intravascular ultrasound (IVUS), is routinely used clinically for assessing blood wall anatomy. In particular, IVUS is used in stent placement, evaluating the state of stent, quantitating arterial remodeling, predicting arterial restenosis and complications following angioplasty and stenting, and characterization of atherosclerotic plaques morphology and composition. IVUS uses high frequencies (>20 MHz) to improve resolution, which allows the delineation of the three layered structures in the vessel wall (adventitia, media and intima); however, with a resolution in excess of 100 microns, it is difficult to resolve early stages of disease such as intimal thickening and fibrous caps. Techniques for performing ultrasonic imaging are known in the art (See Wells, P.N.T., “Ultrasonic Imaging of the Human Body,” Rep. Prod. Phys., 62:671-722 (1999)).

Nuclear magnetic resonance imaging is based on the observation that a proton in a magnetic field has two quantized spin states. In particular, NMR allows for the determination of the structure of organic molecules and allows users to see pictures representing structures of molecules and compounds (I.e., bones, tissues and organs). Groups of nuclei brought into resonance, that is, nuclei absorbing and emitting photons of similar electromagnetic radiation (e.g., radio waves) make subtle yet distinguishable changes when the resonance is forced to change by altering the energy of impacting photons. Techniques for performing NMR imaging are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999)).

Magnetic resonance imaging relies on the principles of atomic nuclear-spin resonance, using strong magnetic fields and radio waves to collect and correlate deflections caused by atoms into images. The technique is used to diagnose or for diagnosis of a broad range of pathologic conditions in all parts of the body including cancer, heart and vascular disease, stroke, and joint and musculoskeletal disorders. Techniques for performing MRI imaging are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999)).

Fluorescence spectroscopy and imaging have the potential to provide information about biochemical, functional and structural changes of bio-molecular complexes in tissues that occur as a result of either pathological transformation or therapeutic intervention (Marcu, L. et al., “Time-resolved Laser-induced Fluorescence Spectroscopy for Staging Atherosclerotic Lesions,” in Fluorescence in Biomedicine, Mycek and Pogue eds., New York: Marcel Dekker (2002); Pasterkamp, G. et al., “Techniques characterizing the coronary atherosclerotic plaque: Influence on clinical decision making,” Journal of the American College of Cardiology 36:13-21 (2000); Lakowicz, J. R. et al., Principles of fluorescence spectroscopy, 2nd ed., New York: Kluwer Academic/Plenum (1999); Andersson-Engels, S. et al., “In vivo fluorescence imaging for tissue diagnostics,” Phys Med Biol 42:815-824 (1997); Bigio, I. J. et al., “Ultraviolet and visible spectroscopies for tissue diagnostics: Fluorescence spectroscopy and elastic-scattering spectroscopy,” Physics in Medicine and Biology 42:803-814 (1997); Cubeddu, R. et al., “Time-resolved fluorescence imaging in biology and medicine,” Journal of Physics D-Applied Physics 35:R61-R76 (2002); Das, B. B. et al., “Time-resolved fluorescence and photon migration studies in biomedical and model random media,” Reports on Progress in Physics 60:227-292 (1997); Drezek, R. A. et al., “Optical imaging of the cervix,” Cancer 98:2015-2027 (2003); Glanzmann, T. et al., “Time-resolved spectrofluorometer for clinical tissue characterization during endoscopy,” Review of Scientific Instruments 70:4067-4077 (1999); Mycek, M. A. et al., “Colonic polyp differentiation using time-resolved autofluorescence spectroscopy,” Gastrointestinal Endoscopy 48:390-394 (1998); Ramanujam, N., “Fluorescence spectroscopy of neoplastic and non-neoplastic tissues,” Neoplasia 2:89-117 (2000); Richards-Kortum, R. et al., “Quantitative optical spectroscopy for tissue diagnosis,” Annual Review of Physical Chemistry 47:555-606 (1996)). Indeed, fluorescence-based devices allow light delivery and collection using fiber optic probes, can facilitate non- or minimally-invasive investigations of tissues with catheters or endoscopic probes, and enhance the diagnostic capability of traditional clinical devices (Id.; Utzinger, U. et al., “Fiber optic probes for biomedical optical spectroscopy,” Journal of Biomedical Optics 8:121-147 (2003)). Fluorescence spectroscopy based techniques have been shown to detect elastin, collagen, lipids and other sources of autofluorescence in normal and diseased arterial walls as well as to characterize the biochemical composition of atherosclerotic plagues both ex vivo and in vivo (Id.; Papazoglou, T. G. et al., “Laser-Induced Fluorescence Detection of Cardiovascular Atherosclerotic Deposits Via Their Natural Emission and Hypocrellin (Ha) Probing,” Journal of Photochemistry and Photobiology B-Biology 22:139-144 (1994); Morguet, A. J. et al., “Autofluorescence spectroscopy using a XeCI excimer laser system for simultaneous plaque ablation and fluorescence excitation,” Lasers Surg Med 14:238-248 (1994); Morguet, A. J. et al., “Development and evaluation of a spectroscopy system for classification of laser-induced arterial fluorescence spectra,” Biomed Tech (Berl) 42:176-182 (1997); Baraga, J. J. et al., “Laser induced fluorescence spectroscopy of normal and atherosclerotic human aorta using 306-310 nm excitation,” Lasers Surg Med 10:245-261 (1990); Bartorelli, A. L. et al., “In vivo human atherosclerotic plaque recognition by laser-excited fluorescence spectroscopy,” J Am Coll Cardiol 17:160B-168B (1991)). In the context of “vulnerable” plaque diagnosis, studies have reported the application of fluorescence techniques to the identification of plaque disruption (Christov, A. et al., “Optical detection of triggered atherosclerotic plaque disruption by fluorescence emission analysis,” Photochem Photobiol 72:242-252 (2000)), detection of plaques with thin fibrous cap (Arakawa, K. et al., “Fluorescence analysis of biochemical constituents identifies atherosclerotic plaque with a thin fibrous cap,” Arterioscler Thromb Vase Biol 22:1002-1007 (2002)), and discrimination of lipid-rich lesions (Marcu, L. et al., “Discrimination of human coronary artery atherosclerotic lipid-rich lesions by time-resolved laser-induced fluorescence spectroscopy,” Arteriosclerosis Thrombosis and Vascular Biology 21:1244-1250 (2001)).

Fluorescence spectroscopy or fluorometry is a type of electromagnetic spectroscopy used for analyzing fluorescent spectra. It involves using a beam of light, usually ultraviolet light, that excites the electrons in molecules of certain compounds and causes them to emit light of a lower energy. Fluorescence spectroscopy techniques can provide detailed surface maps based on differences in chemical composition of tissue fluorescence emission spectra. Fluorescence spectroscopy may also detect and provide information regarding the chemical composition of a sample. Techniques for performing fluorometry are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999); Warren, S. et al., “Combined Ultrasound and Fluorescence Spectroscopy for Physico-Chemical Imaging of Atherosclerosis,” IEEE Transactions on Biomed Engineering 42(2):121-132 (1995); Marcu, L., et al., “In vivo Detection of Macrophages in a Rabbit Atherosclerotic model by Time-resolved Laser-induced Fluorescence Spectroscopy” Atherosclerosis 181:285-303 (2005)).

Raman spectroscopy also involves using a beam of light, usually ultraviolet light, that excites the electrons in molecules of certain compounds and causes them to emit light of a lower energy. Raman spectroscopy is based on the Raman effect, which is the inelastic scattering of photons by molecules. In Raman scattering, the energies of the incident and scattered photons are different. The Raman scattered light occurs at wavelengths that are shifted from the incident light by the energies of molecular vibrations. Typical applications are in structure determination, multicoinponent qualitative analysis, quantitative analysis, and chemical and/or biochemical analysis. Techniques for performing Raman spectroscopy are known in the art (See Ferraro, J., Introductory Raman Spectroscopy, Academic Press, 2^{nd} ed. (2003); McCreery, R., Raman Spectroscopy for Chemical Analysis, John Wiley and Sons, Inc., (2000)).

Near infrared (NIR) spectroscopy is the measurement of the wavelength and intensity of the absorption of near-infrared light by a sample. Near-infrared light spans the 800 nm-2.5 μm range and is energetic enough to excite overtones and combinations of molecular vibrations to higher energy levels. NIR spectroscopy is typically used for quantitative measurement of organic functional groups. Techniques for performing NIR spectroscopy are known in the art (See Hollas, J., Modern Spectroscopy, John Wiley and Sons, Inc., 4^{th} ed. (2004)).

Magnetic resonance spectroscopy (MRS) is the use of the nuclear magnetic resonance phenomenon to study physical, chemical, and biological properties of matter. Nuclear magnetic resonance is a phenomenon which occurs when the nuclei of certain atoms are immersed in a static magnetic field and exposed to a second oscillating magnetic field. MRS produces a characteristic spectrum of a specific nucleus, such as a proton (¹H) or a carbon (¹³C), in which the resonance frequency is influenced by the surrounding environment and neighboring nuclei cause an effect (coupling) on the observed signals. Techniques for performing MRS are known in the art (See Hollas, J., Modern Spectroscopy, John Wiley and Sons, Inc., 4^{th} ed. (2004)).

Reflectance spectroscopy is the study of light as a function of wavelength that has been reflected or scattered from a solid, liquid, or gas. As photons enter a sample, some are reflected, some pass through, and some are absorbed. Those photons that are reflected or refracted through a particle are said to be scattered. Scattered photons may encounter another particle or be scattered away from the surface so they may be detected and measured. Photons are absorbed by several processes. The variety of absorption processes and their wavelength dependence allows information to be derived regarding the chemistry of a sample from its reflected light. Techniques for performing reflectance spectroscopy are known in the art (See Hollas, J., Modern Spectroscopy, John Wiley and Sons, Inc., 4^{th} ed. (2004)).

Laser speckle imaging (LSI) is an optical imaging modality that can be used for functional mapping of a target region. Laser speckle fluctuations in time and space provide information about the local motion close to the surface of laser illuminated turbid objects. Thus one may gain knowledge about fluid dynamics in tissue by computer processing the digital picture of laser illuminated tissue. Laser speckle contrast analysis (LASCA) has been successfully applied to retinal, skin and cerebral blood flow. Laser speckle is a random optical interference effect produced by the coherent addition of scattered laser light with slightly different path lengths. Techniques for performing LSI are known in the art (See Francon, M. Laser Speckle and Application in Optics, Academic Press (1979)).

Computed tomography imaging, also called CT, computed axial tomography or CAT scans, use advanced computer-based mathematical algorithms to combine different reading or views of a patient into a coherent picture usable for diagnosis. CT scans use high energy electromagnetic beams, a sensitive detector mounted on a rotating frame, and digital computing to create detailed images. Techniques for performing CT scans are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999)).

Optical coherence tomography (OCT), is a diagnostic medical and veterinary imaging technology that utilizes photonics and fiber optics to obtain images and tissue characterization information. OCT employs infrared light waves that reflect off the internal microstructure within biological tissues or other suitable targets. OCT delivers infrared light to the imaging site through a single optical fiber, and the imaging guidewire contains a complete lens assembly to perform a variety of imaging functions. Techniques for performing OCT are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999)).

Positron emission tomography allows physicians to measure cell activity in organs, using rings of detectors that surround the patient to track the movements and concentrations of radioactive tracers. The detectors measure gamma radiation produced when positrons emitted by tracers are annihilated during collisions with electrons. PET scans are used to study mental diseases such as schizophrenia and depression, and to measure reactions of the brain to sensory input (e.g., hearing, sight, smell), activities associated with processing information (e.g., learning functions), physiological reactions to addiction, metabolic processes associated with osteoporosis and atherosclerosis, and to shed light on pathological conditions such as Parkinson and Alzheimer's diseases. Techniques for performing PET are known in the art (See Armstrong, P. et al., Diagnostic Imaging, Blackwell Publishing, 5th ed. (2004); Grainger, R. G. and D. J. Allison, Diagnostic Radiology: A Textbook of Medical Imaging, Edinburgh, Scotland: Harcourt Brace, 3rd ed. (1999)).

In many settings, multiple forms of imaging and/or spectroscopy are increasingly used to help physicians increase the speed and accuracy of diagnosis and minimize the need for invasive surgeries. Indeed, the development of accurate, accessible and relatively inexpensive non-invasive technologies has changed the way in which physicians care for patients. Diagnostic imaging and/or spectroscopic tools can be used in conjunction with other therapies known in the art, including stenting and balloon angioplasty, by providing vascular images in real time to guide stent placement and balloon inflation. However, many of these advances have drawbacks; for example, many noninvasive imaging modalities rely on indirect measurements and properly diagnosing a patient may require multiple imaging and/or spectroscopic techniques. Moreover, combined imaging applications are often hampered by an inability to simultaneously correlate imaging data to independently diagnose and treat pathology (See, e.g., Warren, S. et al., “Combined Ultrasound and Fluorescence Spectroscopy for Physico-Chemical Imaging of Atherosclerosis,” IEEE Transactions on Biomed Engineering 42(2):121-132 (1995)). Thus, there remains a need in the art for instrumentation and methods that combine multiple forms of imaging and/or spectroscopy to allow physicians to more accurately and efficiently diagnose and treat patients.

A catheter is a flexible tube inserted into some part of the body that provides a channel for fluid passage or a medical device. A catheter can be thin and flexible (e.g., soft or silastic catheter), or it can be larger and solid (e.g., hard catheter). Placement of a catheter into a particular part of the body may allow for draining urine, fluid collection (e.g., abdominal abscess), administration of intravenous fluids, medication or parenteral nutrition, angioplasty, angiograply, balloon septostomy, direct measurement of blood pressure in an artery or vein. One example is a balloon catheter, which is a tube comprised of rubber or other suitable material with a balloon tip that is inserted into the bladder via the urethra, and filled with sterilized liquid or air. Another example is a central venous catheter, which is a conduit for giving drugs or fluids into a large-bore catheter. Central venous catheters and/or silastic catheters are common tools used for long-term vascular access. Silastic catheters have a variety of uses including collection of fluids, introduction of chemotherapy, measurement of intracranial pressure and imaging of vascular tissues. The catheters are designed with relatively inert and biocompatible materials and offer increased pliability. However, conventional silastic catheters have several shortcomings; for example, an operator wishing to both ultrasonically image and optically image a vascular tissue or other suitable target must remove and replace the catheter for each modality. This may result in one or more complications, including improper location of the catheter upon reinsertion, injury to the surrounding tissues or target area by the stylet and the catheter or injury to the vascular structures or target structure. These problems can also result in increased catheter malfunction, leading to increased infection and clotting rates. Current technologies and methods consist of limited optics-based or ultrasonic-based modalities. (See, e.g., U.S. Pat. No. 5,690,117; U.S. Pat. No. 6,659,957; U.S. Pat. No. 6,193,666; U.S. Pat. No. 5,492,126; U.S. Pat. No. 4,327,738; Tearney, G. J. et al. (2003) Circulation 107:113-119; and Barton, J. K. et al. (2004) J. Biomed Optics 9(3):618-623). Due to the large difference in the frequency content of optics-based and ultrasonic-based modalities, different information is provided during probing of a target area. Techniques for using and constructing catheters are known in the art (See Muhm M., “Ultrasound guided central venous access,” Br Med J., 325:1373-1374 (2002); Polderman, K. H. and Girbes, A. R. J., “Central venous catheter use,” Intensive Care Med., 28:1-28 (2002)).

A biopsy probe is a device used to obtain a sample of a target for examination. Biopsy probes are used in the medical fields to assist in diagnosis of disease conditions; for example, liver biopsy (i.e., hepatitis, cirrhosis), endometrial biopsy (i.e., abnormal bleeding), prostate biopsy (i.e., prostate cancer), skin biopsy (i.e., melanoma), bone marrow biopsy (i.e., diseases of blood and lymphatic systems), breast biopsy (i.e., breast cancer), small intestine biopsy (i.e., coeliac disease). There are many techniques for performing biopsy including, for example, aspiration or FNA biopsy, cone biopsy, core needle biopsy, suction assisted core biopsy, endoscopic biopsy, punch biopsy, surface biopsy, and surgical biopsy. The most appropriate method of biopsy and guidance are determined based on various factors including the tissue, organ or body part to be sampled; the initial diagnosis of the abnormality; the size, shape and other characteristics of the abnormality; the location of the abnormality; the number of abnormalities; other medical conditions of the patient; and the preference of the patient. Generally, biopsies are guided by the method that best identifies the abnormality. For example, palpable lumps can be felt and therefore no additional guidance is needed in most cases. On the other hand, lesions discovered by an imaging test, for example, mammography or CT, will often need biopsy that is guided by the modality that best shows the lesion. Biopsy probes may be directed to a target using any suitable imaging and/or spectroscopic technique. General techniques for using and constructing biopsy probes are known in the art.

A cannula is a flexible tube which when inserted into the body is used either to withdraw fluid or insert medication. Conventional cannulae come with a trocar (a sharp pointed needle) attached which allows puncture of the body to get into an intended space. A push-pull cannula, which both withdraws and injects fluid, can be used to determine the effect of a certain chemical on a specific cell, tissue or area of the body. General techniques for using and constructing cannulae are known in the art.

Endoscope is a general term used to describe a device for viewing specific parts and organs of the body. Endoscopy is a medical and veterinary procedure that allows a practitioner to observe the inside of the body without performing major surgery. An endoscope is a long flexible and/or rigid tube with a lens at one end and a telescope at the other. An endoscope can convey images with a fiber imaging bundle (fiberscope) or a relay of lenses (laparoscope). The end with the lens is inserted into the patient. Light passes down the tube (via bundles of optical fibers or relay of lenses) to illuminate the relevant area and the telescopic eyepiece magnifies the area so the practitioner can see what is there with or without a camera (naked eye). Usually, an endoscope is inserted through one of the body's natural openings, such as the mouth, urethra or anus. Some endoscopies may require a small incision through the skin, and are usually performed under general or local anesthesia. Major types of endoscopy include, for example, gastroscopy, esophagoscopy, colonscopy, cystoscopy, bronchoscopy, laryngoscopy, nasopharyngoscopy, laparoscopy, anthroscopy and thoracoscopy. There are a number of other sub-types of “scopies” including, for example, proctoscopy, sigmoidoscopy, nephro-ureteroscopy, mediastinoscopy, choledochoscopy, angioscopy. Techniques for using and constructing endoscopes are known in the art (See Petelin, J., “Surgical Laparoscopy, Endoscopy & Percutaneous Techniques,” Ambulatory Surgery, 9(4):310 (1999)).

The foregoing examples of the related art and limitations related therewith are intended to be illustrative and not exclusive. Other limitations of the related art will become apparent to those of skill in the art upon a reading of the specification and a study of the drawings. All references cited herein are incorporated by reference as if fully set forth herein.

### SUMMARY OF THE INVENTION

The following embodiments and aspects thereof are described and illustrated in conjunction with systems, tools and methods that are meant to be exemplary and illustrative, not limiting in scope. In various embodiments, one or more of the above-described problems have been reduced or eliminated, while other embodiments are directed to other improvements.

Diagnostic imaging and/or spectroscopic components are used in a variety of probing devices for accurate and efficient diagnosis and treatment. In various embodiments, an apparatus and methods for simultaneously collecting images and spectroscopic information from a cavity are provided. In other embodiments, an apparatus and methods for simultaneously collecting information about the structure and composition of a cavity are provided. In further embodiments, an apparatus and methods for simultaneously ultrasonically imaging, optically imaging and/or obtaining diagnostic spectra of a cavity to analyze an image and to detect chemical composition are provided.

One embodiment by way of non-limiting example includes an apparatus constructed in accordance with this invention with an outer sheath, an inner tube and at least one optical fiber adapted to collect images and spectroscopic information from a cavity. The outer sheath may have a distal end, a proximal end and a longitudinal bore. The inner tube may have a hollow shaft that is configured coaxially within the outer sheath, and the tube may incorporate various imaging and spectoscopic components. For example, the apparatus may incorporate at least one optical fiber. In various embodiments, the optical fiber(s) may be adapted to perform any imaging and/or spectroscopic technique including, for example, fluorescence spectroscopy, optical coherence tomography, laser speckle imaging, Raman spectroscopy, near-infrared spectroscopy, reflectance spectroscopy or a combination thereof. In various embodiments, the apparatus may be configured to collect images and spectroscopic information from one or more regions of interest.

Another embodiment by way of non-limiting example includes the apparatus configured with an ultrasonic transducer and an optical fiber for imaging and/or spectroscopic applications. In other embodiments, the apparatus may optionally be configured with a magnetic resonance spectroscopy coil. In other embodiments, the apparatus may be configured with any suitable imaging and/or spectroscopy means.

Another embodiment by way of non-limiting example includes the apparatus configured with an inlet that extends into the sheath and longitudinal bore for infusion of a solution. Any suitable solution may be infused into the longitudinal bore to lubricate, sterilize and/or irrigate the various components of the apparatus and/or portions of a cavity. In various embodiments, the apparatus may incorporate windows near the distal end of the sheath to allow for fluid communication between the longitudinal bore and a cavity.

Another embodiment by way of non-limiting example includes the apparatus configured with various components for collecting information about the apparatus and a cavity in which it is deployed. In various embodiments, for example, the apparatus may incorporate at least one x-ray marker into the outer sheath and/or inner tube to allow the operator to locate the device and its components in a cavity. In other embodiments, the apparatus may be configured with a thermal wire incorporated throughout the length of the outer sheath for sensing the temperature of a cavity. In other embodiments, the apparatus may be configured with a transluminant dome at the distal end of the inner tube to allow for unimpeded collection of images and/or spectroscopic information. For example, the transluminant dome may be constructed of optical quality silica to allow for the passage of excitation light and collection of diagnostic spectra. In various embodiments, the apparatus may be configured with a light and/or sound wave reflector for aiming light and sound waves within a cavity.

Another embodiment by way of non-limiting example includes the apparatus configured with components that can be modulated. In various embodiments, for example, the inner tube may rotate coaxially within the sheath and/or may move longitudinally with respect to the outer sheath. The inner tube may rotate clockwise or counter-clockwise and may be advanced or withdrawn longitudinally with respect to the outer sheath. In various embodiments, one or more of the imaging and/or spectroscopy components may optionally rotate independently from the rotating inner tube. In various embodiments, the apparatus may include stabilizing rings and nodes to prevent longitudinal movement of the imaging and/or spectroscopy components.

Another embodiment by way of non-limiting example includes the apparatus configured with an imaging modality (IVUS, OCT, angioscopy, laser speckle, intravascular MRI) for collecting structural or anatomic information about a cavity, and a spectroscopy modality (fluorescence, Raman, reflectance, near-infrared, magnetic resonance spectroscopy) for collecting biochemical information (composition) about a cavity and temperature (thermography) in a cavity or structures within cavity.

Another embodiment by way of non-limiting example includes methods of using the multiple modality apparatus to accurately introduce the apparatus into a cavity. In various embodiments, the apparatus and methods prolong catheter life and reduce catheter obstruction. In various embodiments, the apparatus may be configured for use as a microscope, endoscope, probe and/or catheter to allow for simultaneous collection of images and diagnostic spectra from a cavity. In other embodiments, the apparatus may be configured as any suitable probe-like device.

In addition to the exemplary aspects and embodiments described above, further aspects and embodiments will become apparent by reference to the drawings and by study of the following detailed descriptions.

### BRIEF DESCRIPTION OF FIGURES

Exemplary embodiments are illustrated in referenced figures of the drawings. It is intended that the embodiments and figures disclosed herein are to be considered illustrative rather than restrictive.

FIG. 1 depicts a longitudinal cross-section of a design schematic for a multiple modality probe in accordance with an embodiment of the invention. The probe employs an ultrasonic transducer and an optical fiber that serves as a rotational axis, which are coaxially arranged within a flexible, hollow, rotating and moveable inner tube covered by an outer sheath with a longitudinal bore. The ultrasonic transducer has a hole through which the optical fiber tip passes to allow for compact coaxial design. The inner tube incorporates a transluminant dome at the distal end of the probe to allow for unimpeded collection of images and spectroscopic information. The outer sheath incorporates an inlet that extends into the longitudinal bore for infusion of solution, and windows near the distal end of the sheath to allow for fluid communication between the longitudinal bore and a cavity. The probe further incorporates x-ray markers into the sheath and inner tube to allow the operator to locate and modulate the probe while deployed in a cavity. The probe further incorporates a thermal wire, which may be used for sensing the temperature of a cavity. The probe further incorporates a reflector for directing light and sound waves from the ultrasonic transducer and optical fiber. The probe further incorporates a stabilizing ring and nodes to prevent longitudinal movement of the optical fiber.

FIG. 2 depicts a perpendicular cross-section of the design schematic of the multiple modality probe shown in FIG. 1.

FIG. 3 depicts a longitudinal cross-section of a design schematic for a multiple modality probe in accordance with an embodiment of the invention. The probe employs the same features as in FIG. 1 with the exception of the “coaxial” ultrasonic transducer and optical fiber. In this design schematic, the probe employs an optical fiber as the rotational axis and the ultrasonic transducer is affixed to the wall of the inner tube and transluminant dome, along with a reflector for light emanating and collected by the optical fiber. In this design schematic, the ultrasonic transducer is attached to image the same or spatially related region of interest as the optical reflecting component.

FIG. 4 depicts a perpendicular cross-section of the design schematic of the multiple modality probe shown in FIG. 3.

FIG. 5 depicts a longitudinal cross-section of a design schematic for a multiple modality probe in accordance with an embodiment of the invention. The probe employs the same features and arrangement as in FIG. 3 with the exception of the target region(s) of interest for collection of images and spectroscopic information. In this design schematic, the ultrasonic transducer is affixed to the wall of the inner tube and transluminant dome to image different or spatially uncorrelated region(s) of interest as the optical reflecting component. The fixed angular orientation between the ultrasonic transducer and the light emanating and collected by the optical fiber is such that the ultrasonic transducer and the optical fiber are directed at multiple regions of interest at fixed angles from the other, for example, targets on opposing areas of a cavity.

FIG. 6 depicts a perpendicular cross-section of the design schematic of the multiple modality probe shown in FIG. 5.

FIG. 7 depicts a longitudinal cross-section of a design schematic for a multiple modality probe in accordance with an embodiment of the invention. The probe employs the same features and arrangement as in FIG. 5 with the exception of the target region(s) of interest for collection of images and spectroscopic information. In this design schematic, the optical fiber may optionally rotate independently of the rotating tube to image different or spatially uncorrelated region(s) of interest. The angular orientation between the ultrasonic transducer and the light emanating and collected by the optical fiber is such that the ultrasonic transducer and the optical fiber are directed at multiple regions of interest at any angle from the other, for example, targets on opposing areas of a cavity, a target in the same area of a cavity, or multiple targets at any angle between.

FIG. 8 depicts a perpendicular cross-section of the design schematic of the multiple modality probe shown in FIG. 7.

FIG. 9 illustrates a probe as depicted in the design schematics of the multiple modality probe shown in FIGS. 1-8 deployed in the femoral vein of a human patient.

### DETAILED DESCRIPTION OF THE INVENTION

All references cited herein are incorporated by reference as if fully set forth herein.

For the embodiments discussed herein, a probing device is provided which may be configured for a variety of functions. The device may be assembled from a variety of materials and may incorporate a variety of features that are based on the specific application of the device. Throughout the application, when one particular illustrative and exemplary embodiment is discussed, any other illustrative and exemplary embodiment may optionally be substituted. In addition to the illustrative and exemplary aspects and embodiments discussed herein, further aspects and embodiments will become apparent by reference to the Figures and by study of the following detailed description.

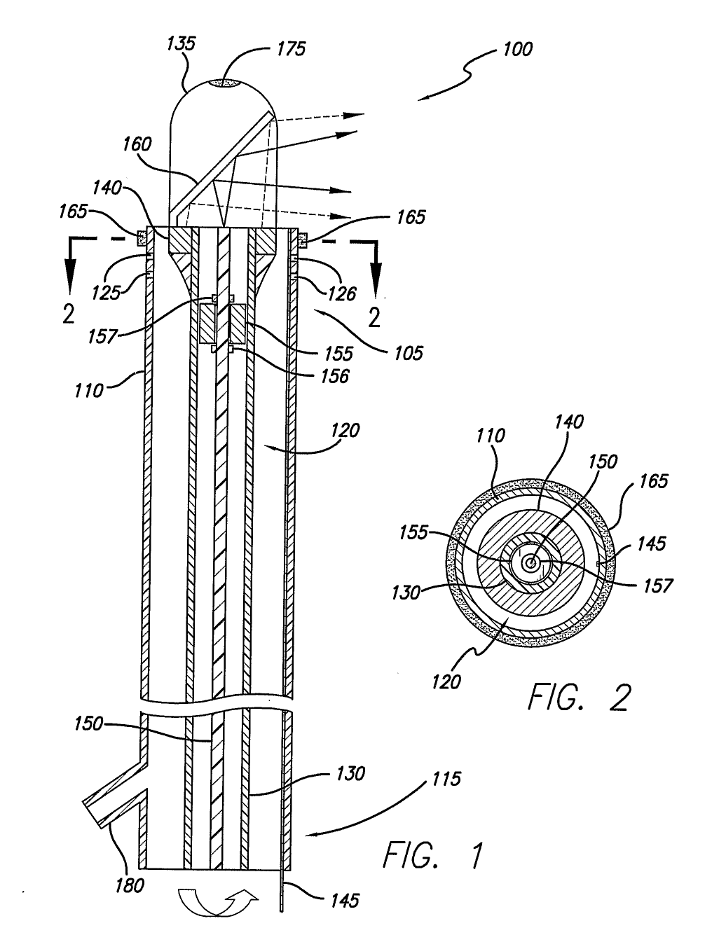

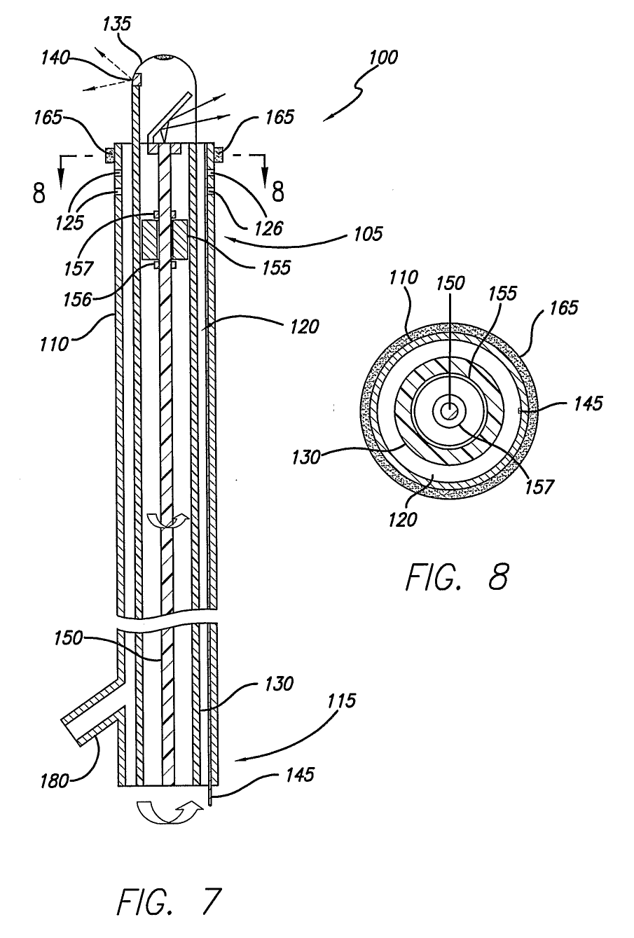

As depicted in FIGS. 1, 3, 5 and 7 as a longitudinal cross-sectional view, the device 100 may be configured with a portion to be inserted into a cavity or hollow space within a mass in which there is a target region of interest (“distal end”) 105, and another portion of which may be configured to remain exterior of the cavity or space when the device is in use (“proximal end”) 115. FIGS. 2, 4, 6 and 8 depict a perpendicular cross-sectional view of the device 100 shown in FIGS. 1, 3, 5 and 7.

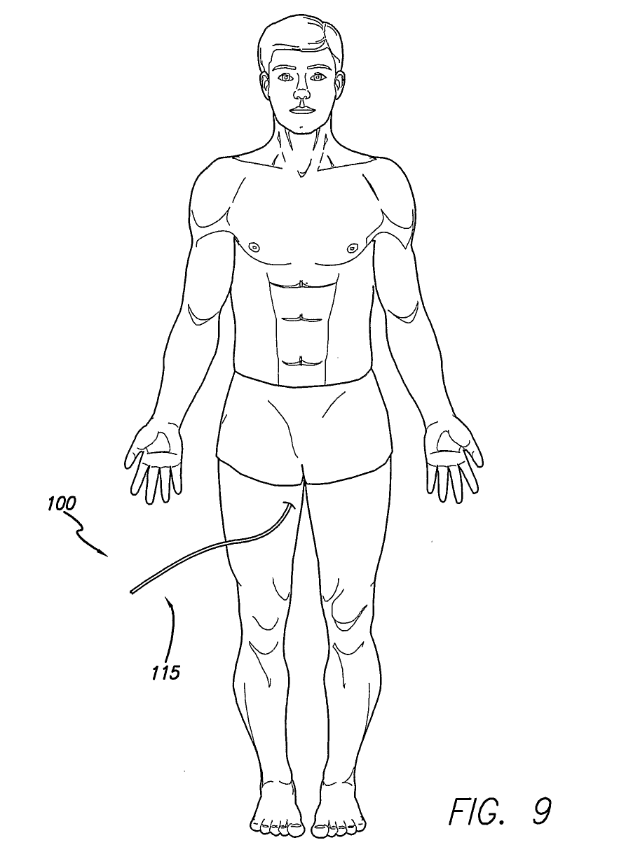

In various embodiments, the device 100 may be configured to be inserted into various systems, including, for example, but in no way limited to the vascular system of a patient, an open cavity, a surgical cavity, other hollow spaces, and combinations thereof. In the embodiments discussed herein, the device 100 may optionally be configured for insertion into areas of the vascular system including, for example, but in no way limited to the subclavian, internal jugular, or femoral veins, other suitable areas of the vascular system, and combinations thereof. As depicted in FIG. 9, the device is deployed in the femoral vein of a human patient. In various embodiments, the device 100 may optionally be configured for insertion into areas of the body including, for example, but in no way limited to the stomach, colon, bladder, large intestine, small intestine, lung, oral cavity, gastrointestinal track, pulmonary tree, brain ventricles, a surgical incision, a surgical cavity, other suitable areas of the body, and combinations thereof. In various embodiments, the device 100 may optionally be configured for insertion into other hollow cavities including, for example, but in no way limited to a pipe or other industrial cavity, tree, ditch, crawl-space, other suitable hollow cavities or hollow spaces within a mass, and combinations thereof. An area is “suitable” if the device 100 can be deployed and advanced into the cavity or area using conventional techniques known to those of ordinary skill in the art.

In various embodiments, the device 100 may be adapted for use as an endoscope, microscope, hand held probing device, biopsy probe or catheter for collection of images and/or spectroscopic information. In one embodiment, the device 100 is adapted for use as a catheter for collection of images and/or spectroscopic information of a tissue or other suitable target region of interest. In various embodiments, the device 100 may be deployed for other uses in conjunction with imaging and spectroscopy including, for example, but in no way limited to draining urine, fluid collection, administration of intravenous fluids, medication or parenteral nutrition, angioplasty, angiography, balloon septostomy, direct measurement of blood pressure in an artery or vein, and combinations thereof.

In various embodiments, the device 100 may include an outer sheath 110 and may have a longitudinal bore 120 (“hollow shaft”) throughout the length of the device 100. The device 100 may have an inner tube 130 that is deployed within the longitudinal bore 120 throughout the length of the device. The longitudinal bore 120 may be of any suitable diameter to accommodate the inner tube 130 as well as the size, function and application of the device 100. The inner tube 130 may have a hollow shaft that incorporates imaging and/or spectroscopic modalities based on the function and application of the device 100. A suitable diameter of the inner tube 130 may be determined by the size, function and application of the imaging and/or spectroscopic modalities included therein, as well as the anatomy of the target region of interest. In various embodiments, an optical quality transluminant dome 135 may be fused to the distal end of the inner tube 130 to allow for unimpeded collection of images and/or spectroscopic information by the imaging and/or spectroscopic modalities of the device 100.

In various embodiments, the device 100 may be flexible and silastic. In other embodiments, the device 100 may be minimally flexible and hard or rigid. The outer sheath 110 and the inner tube 130 may be constructed of any conventional material, as will be readily appreciated by those of skill in the art; for example, various plastics may be used to construct the outer sheath 110 and the inner tube 130 of the device 100 including alloys, silicone and the like. The transluminant dome 135 may be constructed of any conventional material, as will be readily appreciated by those of skill in the art including, for example, any optical quality plastic, polymer, silica and the like. In various embodiments, the conventional material may be medical grade. Other materials will be readily recognized by one of ordinary skill in the art, and therefore are included herein.

In various embodiments, the device 100 may optionally be configured for use with a guide to insert and/or position the device 100 within the cavity. The guide may be any conventional guide used to deploy probing devices including, for example, but in no way limited to an insertion needle, cannulae, wire, hose, tube, and combinations thereof. Other guides will be readily recognized by one of ordinary skill in the art, and therefore are included herein. In other embodiments, the device 100 may incorporate x-ray opaque markers to allow for tracking the device 100 with fluoroscopy (x-rays). For example, the outer sheath 110 and the inner tube 130 may optionally incorporate x-ray opaque markers 165, 175 that are distinguishable from one another such that the device 100 operator may determine the insertion length of the outer sheath 110 and the inner tube 130 and/or may determine the angular orientation of each of the imaging and/or spectroscopic modalities within the inner tube 130. As depicted in FIGS. 1, 3, 5 and 7, the outer sheath 110 and the transluminant dome 135 may incorporate x-ray opaque markers 165, 175. Other configurations of the x-ray opaque markers 165, 175 will be readily apparent to one of ordinary skill in the art and therefore are included herein.

In various embodiments, the inner tube 130 may optionally rotate on a parallel axis to the longitudinal bore 120 and may be advanced and/or withdrawn along a parallel axis to the longitudinal bore 120 (“longitudinal movement”). Rotating, advancing and/or withdrawing the inner tube 130 may be accomplished using any conventional technique including, for example, but in no way limited to bearings, cable(s), and combinations thereof. Other techniques will be readily recognized by one of ordinary skill in the art, and therefore are included herein. The inner tube 130 may be rotated, advanced and/or withdrawn based on the application of the device 100, the type of imaging and/or spectroscopic modalities included therein, and the cavity and/or hollow space in which the device 100 is inserted. In other embodiments, the imaging and/or spectroscopic modalities included in the device 100 may optionally rotate independently of the inner tube 130. Rotation of the imaging and/or spectroscopic modalities and the inner tube 130 may be independent and/or coordinated and may be clockwise and/or counterclockwise.

In various embodiments, the inner tube 130 of the device 100 may be configured to contain an imaging and/or spectroscopic component to allow for the collection of images and/or spectroscopic information from the cavity and/or hollow space. For example, the imaging and/or spectroscopic component may be an ultrasonic component, endoscopic component, electromagnetic component, magnetic resonance imaging (MRI) component, x-ray component, fluorescence component, nuclear magnetic resonance (NMR) component, computed tomography (CT) component, positive emission tomography (P.E.T.) component, optical component, laser speckle component, Raman spectroscopic component, near infrared (NIR) spectroscopic component, magnetic resonance spectroscopic (MRS) component, reflectance spectroscopic component and combinations thereof.

In various embodiments, the inner tube 130 of the device 100 may be configured to contain a second imaging and/or spectroscopic component to allow for the collection of images and/or spectroscopic information from the cavity and/or hollow space. For example, the second imaging and/or spectroscopic component may be an ultrasonic component, endoscopic component, electromagnetic component, magnetic resonance imaging (MRI) component, x-ray component, fluorescence component, nuclear magnetic resonance (NMR) component, computed tomography (CT) component, positive emission tomography (P.E.T.) component, optical component, laser speckle component, Raman spectroscopic component, near infrared (NIR) spectroscopic component, magnetic resonance spectroscopic (MRS) component, reflectance spectroscopic component and combinations thereof.

As depicted in FIGS. 1-8, the device 100 may be configured to contain an ultrasonic component to allow for indirect imaging. This allows the device 100 operator to sonically direct the device 100. A variety of ultrasonic transducers, for example, are known in the art and may be suitable for this purpose. As depicted in FIGS. 1-8, an ultrasonic transducer 140 may be any suitable ultrasonic transducer based on the size, function and application of the device 100. For example, an ultrasonic transducer 140 that is capable of transmitting sound waves and receiving reflected sound waves may be used.

As depicted in FIGS. 1-8, the device 100 may be configured to contain an optical component for the collection of direct optical images and/or spectroscopic information. This may allow the device 100 operator to, for example, optically verify the correct placement of the device 100, deploy excitation laser light and/or collect fluorescent data. A variety of optical fibers, for example, are known in the art and may be suitable for this purpose. As depicted in FIGS. 1-8, an optical fiber 150 may be any suitable optical catheter based on the size, function and application of the device 100. For example, an optical fiber 150 used for illuminating and image conducting. In various embodiments, an optical fiber 150 may be used for both excitation and collection of fluorescence (dichroic beam splitter arrangement). The optical fiber 150 may be assembled from a concentrically-arranged layer of illuminating fibers that carry light to the field of view from a light source, and a central image conducting core which transmits the image from the field of view to a camera, video monitor, and/or related electronics and hardware.

In various embodiments, as depicted in FIG. 1, the device 100 may contain a light and sound wave reflector 160 that is used to aim the light and sound waves from the ultrasonic transducer 140 and the optical fiber 150. For example, as depicted in FIG. 1, the device 100 may be configured such that the tip of the optical fiber 150 passes through a central hole in the ultrasonic transducer 140 (“coaxial arrangement”). In this coaxial arrangement, light and sound waves from the ultrasonic transducer 140 and the optical fiber 150 are directed towards the same or spatially correlated target region of interest using the light and sound wave reflector 160, as indicated by the solid line (optical image) and dashed line (ultrasonic image) arrows emanating from the ultrasonic transducer 140 and the optical fiber 150. The phrase “same or spatially correlated” as used herein refers to target areas that substantially coincide. The central hole may be any suitable diameter to accommodate the distal diameter of the optical fiber 150. The device 100, as depicted in FIG. 1, may allow for more compact design (i.e. smaller outer diameter), which may be beneficial when deployed in a cavity or hollow space.

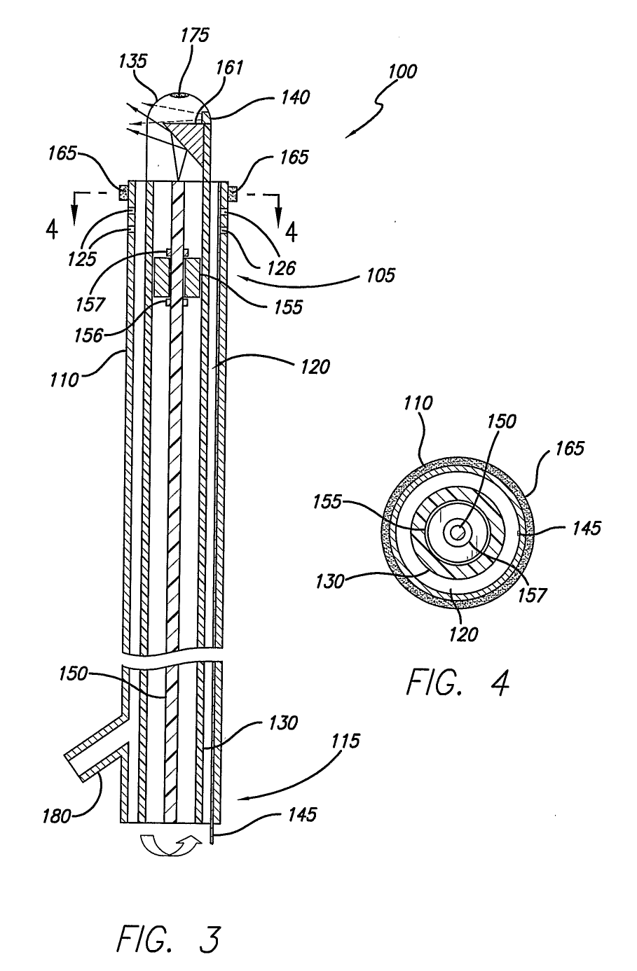

In other embodiments, as depicted in FIGS. 3, 5 and 7, the device 100 may be configured with a light reflector 161 that works in conjunction with the optical fiber 150 and the ultrasonic transducer 140 may be configured to be used without a separate sound wave reflector. For example, as depicted in FIGS. 3, 5 and 7, the device 100 may be configured such that the optical fiber 150 and the ultrasonic transducer 140 are not coaxial; for example, the ultrasonic transducer 140 may be attached to the transluminant dome 135 fused to the distal end of the inner tube 130. As depicted in FIG. 3, the ultrasonic transducer 140 and optical fiber 150 may be arranged on separate axes and target the same or spatially correlated areas, as indicated by the solid line (optical image) and dashed line (ultrasonic image) arrows emanating from the ultrasonic transducer 140 and the optical fiber 150.

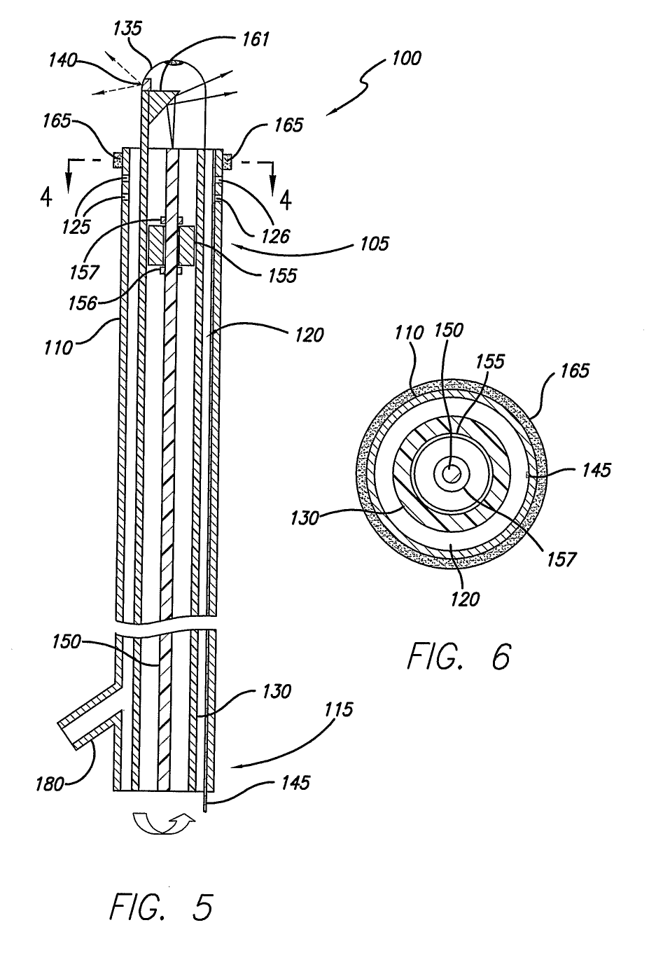

In other embodiments, as depicted in FIG. 5, the ultrasonic transducer 140 and the optical fiber 150 may be arranged on separate axes and target different or spatially uncorrelated areas at a fixed angle, as indicated by the solid line (optical image) and dashed line (ultrasonic image) arrows. In this way, the ultrasonic transducer and optical probe may be arranged to target multiple regions of interest, which are related only by the pre-determined angular orientation of the imaging and/or spectroscopic modalities of the device 100. The phrase “different or spatially uncorrelated” as used herein refers to target areas that do not substantially coincide.

In other embodiments, as depicted in FIG. 7, the ultrasonic transducer 140 and the optical fiber 150 may be arranged on separate axes and target different or spatially uncorrelated areas at any angle, as indicated by the solid line (optical image) and dashed line (ultrasonic image) arrows. This may be accomplished, as depicted in FIG. 7, where the optical fiber 150 may optionally rotate on a parallel axis to the inner tube 130 and the longitudinal bore 120. Rotation of the optical fiber 150 and the inner tube 130 may be independent and/or coordinated and may be clockwise and/or counterclockwise. As depicted in FIG. 7, the rotating inner tube 130 and the rotating optical fiber 150 may allow for simultaneous rotation of both the optical component and ultrasonic component of the device 100. In this way, the ultrasonic transducer 140 and optical fiber 150 may be arranged to target multiple regions of interest.

In various embodiments, the inner tube 130 may have a ring 155 fused inside the distal end of the inner tube 130 to stabilize the optical fiber 150 during rotation and during movement of the inner tube 130. In various embodiments, nodes 156, 157 may be fused to the optical fiber 150 on either side of the ring 155 to prevent the optical fiber 150 from moving parallel to the inner tube 130. Any number of rings and nodes may be used to stabilize the optical fiber 150. Other techniques for stabilizing the optical fiber 150 will be readily recognized by one of ordinary skill in the art, and therefore are included herein.

In various embodiments, a computer workstation will control the imaging and/or spectroscopic modalities using an instrument control/interface software for data acquisition and analysis. Any suitable computer workstation may be used to control the imaging and/or spectroscopic modalities, as will be appreciated by one of skill in the art. Other techniques for controlling the imaging and/or spectroscopic modalities will be readily recognized by one of ordinary skill in the art, and therefore are included herein.

In various embodiments, the device 100 may provide for simultaneous collection of low and high spatial resolution images of a target(s) using conventional modalities. In other embodiments, the device 100 may provide for simultaneous collection of images and/or spectroscopic information (to detect chemical composition(s)) of a target(s) using conventional modalities. In one embodiment, as depicted in FIG. 1, the device 100 may be configured such that the ultrasonic transducer 140 and the optical fiber 150 work in conjunction with the other using the light and sound wave reflector 160. For example, the ultrasonic transducer 140 and the optical fiber 150 are arranged and correlated to use the light and sound wave reflector 160 to target the same or spatially correlated region of interest. In another embodiment, as depicted in FIG. 3, the ultrasonic transducer 140 and the optical fiber 150 are arranged and correlated such that the ultrasonic transducer 140 directly targets a region of interest and the optical fiber 150 targets the same or spatially correlated region of interest using the light reflector 161. In various embodiments, the ultrasonic transducer 140 and the optical fiber 150 are arranged and correlated to target different or spatially uncorrelated regions of interest. For example, as depicted in FIGS. 5 and 7, the ultrasonic transducer 140 and the optical fiber 150 are arranged and correlated such that the ultrasonic transducer 140 directly targets a region of interest and the optical fiber 150 targets a different or spatially uncorrelated region of interest using the light reflector 161. Other configurations of the device 100 will be readily apparent to one of ordinary skill in the art and therefore are included herein.

In various embodiments, the device 100 may incorporate an inlet 180 near the proximal end of the outer sheath 110 to allow for the administration of any conventional solution into the longitudinal bore 120. In one embodiment, the device 100 may be configured to allow the solution to clear the insertion end optics. In another embodiment, the device 100 may be configured to allow the solution to provide lubrication for the longitudinal bore 120. In another embodiment, the device 100 may be configured with one or more windows 125, 126 near the distal end of the device 100, which allow for fluid communication between the longitudinal bore 120 and the cavity in which the device 100 is deployed. A variety of solutions and/or fluids may be infused into the inlet 180 for irrigation, lubrication and/or sterilization. For example, the inlet 180 may be infused with any conventional solution including, for example, but in no way limited to sterile saline, lubricant, oil, distilled water, other suitable fluids, and combinations thereof. In various embodiments, the fluid may be medical grade. Other conventional solutions will be readily recognized by one of ordinary skill in the art, and therefore are included herein.

In various embodiments, the device 100 may optionally incorporate a thermal wire 145 for temperature sensing. In one embodiment, the thermal wire 145 may be incorporated into the structure of the outer sheath 110. Any conventional material may be used to construct the thermal wire 145 as will be appreciated by one of skill in the art. In one embodiment, the material used to construct the thermal wire 145 may be medical grade copper. Other conventional materials will be readily recognized by one of ordinary skill in the art, and therefore are included herein.

The device discussed in various embodiments herein combines multiple imaging and/or spectroscopic modalities in one delivery system such as a catheter, endoscope, microscope, or hand held probe. The device of the present invention is minimally invasive and allows for simultaneous collection of images and/or spectroscopic information from a region of interest and/or multiple regions of interest, thereby allowing its operator to simultaneously view low and high spatial resolution images and/or spectroscopic information (to detect chemical compositions) from the region(s) of interest. In the context of atherosclerosis and vulnerable plaque detection, the embodiments discussed herein incorporate structural definition of a high-resolution modality, such as OCT, intravascular MRI or high-frequency IVUS, with biochemical processes detected by spectroscopy and thermography. For example, a catheter-based diagnostic device that combines two complementary technologies—optical spectroscopy (time-resolved fluorescence) and ultrasonography (high frequency IVUS)—for the characterization and diagnosis of vulnerable plaques. Indeed, cardiovascular studies demonstrate the need for sensitive and accurate techniques for detection of structural characteristics (morphology) or/and functional properties (activity) associated with rupture-prone plaques. Moreover, a combination of spectroscopic techniques (fluorescence, near-infrared, Raman, reflectance, magnetic resonance) with imaging techniques (IVUS, OCT, angioscopy, laser speckle) may provide greater diagnostic value than each of these techniques alone. Such combination may be useful for both intravascular clinical diagnostic of vulnerable plaque as well as in monitoring the effects of pharmacologic intervention.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. An apparatus for simultaneously collecting images and spectroscopic information from a cavity, comprising:
an outer sheath having a distal end, a proximal end and a longitudinal bore;
an inner tube having a hollow shaft, wherein said inner tube is configured coaxially within said sheath; and
at least one optical fiber within said tube, wherein said optical fiber is adapted to collect images and spectroscopic information from a cavity.

2. The apparatus of claim 1, further comprising an ultrasonic transducer within said tube, wherein said ultrasonic transducer is adapted to collect images from said cavity.

3. The apparatus of claim 1, further comprising a magnetic resonance spectroscopy coil within said tube, wherein said coil is adapted to collect spectroscopic information from said cavity.

4. The apparatus of claim 1, further comprising an inlet extending into said sheath and said longitudinal bore at the proximal end of said sheath and said longitudinal bore, wherein said inlet is adapted to enable the infusion of a solution into said longitudinal bore.

5. The apparatus of claim 1, further comprising at least one window extending into said sheath and said longitudinal bore at the distal end of said sheath and said longitudinal bore, wherein said window is adapted to provide fluid communication between said longitudinal bore and said cavity.

6. The apparatus of claim 1, further comprising at least one x-ray marker incorporated at the distal end of said sheath, wherein said marker is adapted for locating said sheath within said cavity.

7. The apparatus of claim 1, further comprising at least one x-ray marker incorporated at the distal end of said tube, wherein said marker is adapted for locating said tube within said cavity.

8. The apparatus of claim 1, further comprising a thermal wire incorporated throughout the length of said sheath, wherein said wire is adapted for sensing the temperature within said cavity.

9. The apparatus of claim 1, wherein said tube is configured with a transluminant dome at the distal end of said tube to allow for the collection of images and spectroscopic information.

10. The apparatus of claim 1, wherein said tube is adapted to rotate within said sheath.

11. The apparatus of claim 1, wherein said tube is adapted to move longitudinally within said sheath.

12. The apparatus of claim 1, wherein said optical fiber is adapted to perform a technique selected from the group consisting of fluorescence spectroscopy, near-infrared spectroscopy, reflectance spectroscopy, Raman spectroscopy, optical coherence tomography, laser speckle imaging, and a combination thereof.

13. The apparatus of claim 1, further comprising a light and sound wave reflector configured to aim light and sound waves within said cavity.

14. The apparatus of claim 1, further comprising a ring secured within said inner tube and around said optical fiber, wherein said ring is adapted to stabilize said optical fiber.

15. The apparatus of claim 14, further comprising nodes secured to said optical fiber on both sides of said ring, wherein said nodes are adapted to prevent longitudinal movement of said optical fiber.

16. The apparatus of claim 2, wherein said optical fiber and said ultrasonic transducer are configured to collect images and spectroscopic information from the same or spatially correlated region.

17. The apparatus of claim 2, wherein said optical fiber and said ultrasonic transducer are configured to collect images and spectroscopic information from different or spatially uncorrelated regions.

18. The apparatus of claim 1, wherein said optical fiber is adapted to rotate within said tube.

19. The apparatus of claim 1, wherein said apparatus is adapted for use as a microscope, an endoscope, a hand held probe, a catheter or combinations thereof.

20. The apparatus of claim 1, wherein said apparatus is adapted for use as a catheter.

21. The apparatus of claim 20, wherein said catheter is adapted for insertion into a cavity of a patient.

22. An apparatus for simultaneously collecting images and spectroscopic information from a cavity, comprising:
an outer sheath having a distal end, a proximal end and a longitudinal bore;
an inner tube having a hollow shaft, wherein said tube is configured coaxially within said sheath;
at least one imaging means within said tube to collect images from a cavity; and
at least one spectroscopy means within said tube to collect spectroscopic information from said cavity.

23. The apparatus of claim 22, wherein said imaging means and said spectroscopy means are configured as an optical fiber.

24. The apparatus of claim 23, wherein said optical fiber is adapted to perform a technique selected from the group consisting of fluorescence spectroscopy, near-infrared spectroscopy, reflectance spectroscopy, Raman spectroscopy, optical coherence tomography, laser speckle imaging, and a combination thereof.

25. The apparatus of claim 22, wherein said imaging means is an ultrasonic transducer.

26. The apparatus of claim 22, wherein said spectroscopy means is a magnetic resonance spectroscopy coil.

27. The apparatus of claim 22, further comprising a thermal wire incorporated throughout the length of said sheath, wherein said wire is adapted for sensing the temperature within said cavity.

28. The apparatus of claim 22, wherein said tube is adapted to rotate within said sheath.

29. The apparatus of claim 22, wherein said tube is adapted to move longitudinally within said sheath.

30. The apparatus of claim 22, further comprising a light and sound wave reflector configured to aim light and sound waves within said cavity.

31. A method of simultaneously collecting images and spectroscopic information from a cavity, comprising:
inserting a portion of an apparatus into a cavity, wherein the apparatus comprises an outer sheath having a distal end, a proximal end and a longitudinal bore; an inner tube having a hollow shaft, wherein said inner tube is configured coaxially within said sheath; and at least one optical fiber within said tube, wherein said optical fiber is adapted to collect images and spectroscopic information from a cavity; and
using the apparatus to simultaneously collect images and spectroscopic information from said cavity.

32. The method of claim 31, wherein said apparatus further comprises an ultrasonic transducer within said tube to collect images from said cavity.

33. The method of claim 31, wherein said apparatus further comprises a magnetic resonance spectroscopy coil within said tube to collect spectroscopic information from said cavity.

34. The method of claim 31, wherein said apparatus further comprises a thermal wire incorporated throughout the length of said sheath for sensing the temperature within said cavity.

35. The method of claim 31, wherein said tube is adapted to rotate within said sheath.

36. The method of claim 31, wherein said tube is adapted to move longitudinally within said sheath.

37. The method of claim 31, wherein said optical fiber is adapted to perform a technique selected from the group consisting of fluorescence spectroscopy, near-infrared spectroscopy, reflectance spectroscopy, Raman spectroscopy, optical coherence tomography, laser speckle imaging, and a combination thereof.

38. The method of claim 31, wherein said apparatus further comprises a light and sound wave reflector configured to aim light and sound waves within said cavity.

39. The method of claim 31, wherein said optical fiber is adapted to rotate within said tube.
